(19) United States
(12) Patent Application Publication
(10) Pub. No.: US 2023/0176020 A1
(43) Pub. Date: Jun. 8, 2023

(54) AIR POLLUTION/ALLERGEN EXPOSURE MEASUREMENT

(71) Applicant: Carrier Corporation, Palm Beach Gardens, FL (US)

(72) Inventors: Holly Dunn, Indianapolis, IN (US); Holly Rhodes, Carmel, IN (US); Francine Dash, Indianapolis, IN (US); Jeremy Riffle, Carmel, IN (US); Alissa D. Doose, Denver, CO (US); Bridget Volz, Lizton, IN (US); Michael J. Birnkrant, Manlius, NY (US)

(21) Appl. No.: 18/076,261

(22) Filed: Dec. 6, 2022

Related U.S. Application Data
(60) Provisional application No. 63/286,328, filed on Dec. 6, 2021.

(51) Int. Cl. G01N 33/00; G01C 21/20

(52) U.S. Cl. CPC G01N 33/0027; G01C 21/20

(57) ABSTRACT

An air pollution/allergen exposure measurement system is provided and includes at least one fixed air pollution/allergen measurement device(s), a wearable article to be worn by a user and comprising a wearable air pollution/allergen measurement device, a mobile device carried by the user for location monitoring of the user and a processing unit. The processing unit is coupled to the fixed air pollution/allergen measurement device(s), the wearable air pollution/allergen measurement device and the mobile device. The processing unit is configured to determine the fixed air pollution/allergen measurement device(s) the user has been within range of from the location monitoring, to determine a quality of air surrounding the user from readings of the wearable air pollution/allergen measurement device and the fixed air pollution/allergen measurement device(s) the user has been within range of and to make recommendations to the user for improving the quality of air surrounding the user.

### CROSS-REFERENCE TO RELATED APPLICATIONS

This application claims priority to U.S. Provisional Application Ser. No. 63/286,328, filed Dec. 6, 2021, the contents of which are hereby incorporated by reference in its entirety.

### BACKGROUND

The present disclosure relates to air pollution/allergen exposure measurement and, more particularly, to methods and systems for indoor and outdoor air pollution/allergen exposure measurements.

Air pollution/allergen is the presence of substances in the atmosphere that are harmful to the health of humans and other living beings, or cause damage to the climate or to materials. There are many different types of air pollutants, such as gases (including ammonia, carbon monoxide, sulfur dioxide, nitrous oxides, methane, carbon dioxide and chlorofluorocarbons), particulates (both organic and inorganic) and biological molecules. Air pollution/allergens may cause diseases, allergies and even death to humans; it may also cause harm to other living organisms such as animals and food crops, and may damage the natural environment (for example, climate change, ozone depletion or habitat degradation) or built environment (for example, acid rain). Both human activity and natural processes can generate air pollution.

### BRIEF DESCRIPTION

According to an aspect of the disclosure, an air pollution/allergen exposure measurement system is provided and includes at least one fixed air pollution/allergen measurement device, a wearable article to be worn by a user and comprising a wearable air pollution/allergen measurement device, a mobile device carried by the user for location monitoring of the user and a processing unit. The processing unit is coupled to the fixed air pollution/allergen measurement devices, the wearable air pollution/allergen measurement device and the mobile device. The processing unit is configured to determine the fixed air pollution/allergen measurement devices the user has been within range of from the location monitoring, to determine a quality of air surrounding the user from readings of the wearable air pollution/allergen measurement device and the fixed air pollution/allergen measurement devices the user has been within range of and to make recommendations to the user for improving the quality of air surrounding the user.

In accordance with additional or alternative embodiments, the fixed air pollution/allergen measurement devices are deployed in a defined space through which the user is movable.

In accordance with additional or alternative embodiments, the fixed air pollution/allergen measurement devices are deployed in at least one of a building through which the user is movable and an outdoor space through which the user is movable.

In accordance with additional or alternative embodiments, the wearable article includes a selectively activatable air quality improvement element and the selectively activatable air quality improvement element is activatable by the user or the processing unit.

In accordance with additional or alternative embodiments, the mobile device includes a global positioning system (GPS) unit for user location monitoring and tracking.

In accordance with additional or alternative embodiments, the mobile device includes at least one of a mobile computing device, a smartphone and a smartwatch and the processing unit is embodied in the mobile device.

In accordance with additional or alternative embodiments, the processing unit is wirelessly coupled to the fixed air pollution/allergen measurement devices and the wearable air pollution/allergen measurement device.

In accordance with additional or alternative embodiments, the mobile device includes a graphical user interface (GUI) by which the processing unit makes the recommendations to the user.

In accordance with additional or alternative embodiments, the processing unit displays, by way of the GUI, at least one of a recommended route for improved air quality by way of the GUI and an air quality goal for the user along with an experienced air quality reading for the user.

According to an aspect of the disclosure, an air pollution/allergen exposure measurement system is provided and includes a wearable article to be worn by a user and including a wearable air pollution/allergen measurement device and a selectively activatable air quality improvement element and a processing unit coupled to the wearable air pollution/allergen measurement device and the selectively activatable air quality improvement element. The processing unit is configured to determine a quality of air surrounding the user from readings of the wearable air pollution/allergen measurement device and to selectively activate the selectively activatable air quality improvement element accordingly for improving the quality of air surrounding the user.

In accordance with additional or alternative embodiments, the selectively activatable air quality improvement element is activatable by the user or the processing unit.

In accordance with additional or alternative embodiments, the processing unit is embodied in at least one of a mobile computing device, a smartphone and a smartwatch carried by the user.

In accordance with additional or alternative embodiments, the processing unit is interacted with by the user by way of a graphical user interface (GUI) of the at least one of the mobile computing device, the smartphone and the smartwatch.

In accordance with additional or alternative embodiments, the processing unit is wirelessly coupled to the wearable air pollution/allergen measurement device and the selectively activatable air quality improvement element.

According to an aspect of the disclosure, an air pollution/allergen exposure measurement method is provided and includes measuring air pollution/allergen at fixed locations, measuring air pollution/allergen surrounding a user, monitoring a location of the user, determining the fixed locations where the user has been from the monitoring of the location, determining a quality of air surrounding the user from the measuring of air pollution/allergen surrounding the user and the measuring of air pollution/allergen at the fixed locations and making recommendations to the user for improving the quality of air surrounding the user.

In accordance with additional or alternative embodiments, the fixed locations are deployed in a defined indoor or outdoor space through which the user is movable.

In accordance with additional or alternative embodiments, the method further includes selectively activating an air quality improvement element for the user.

In accordance with additional or alternative embodiments, the monitoring of the location of the user includes global positioning of the user.

In accordance with additional or alternative embodiments, the making the recommendations includes making the recommendations via a graphical user interface (GUI).

In accordance with additional or alternative embodiments, the making the recommendations via the GUI includes at least one of recommending a route for improved air quality and displaying an air quality goal for the user along with an experienced air quality reading for the user.

Additional features and advantages are realized through the techniques of the present disclosure. Other embodiments and aspects of the disclosure are described in detail herein and are considered a part of the claimed technical concept. For a better understanding of the disclosure with the advantages and the features, refer to the description and to the drawings.

### BRIEF DESCRIPTION OF THE DRAWINGS

For a more complete understanding of this disclosure, reference is now made to the following brief description, taken in connection with the accompanying drawings and detailed description, wherein like reference numerals represent like parts:

FIG. 1 is a schematic illustration of an exemplary air pollution/allergen exposure measurement system in accordance with embodiments;

FIG. 2 is a schematic diagram illustrating components of an exemplary processing unit of the air pollution/allergen exposure measurement system of FIG. 1 in accordance with embodiments;

FIG. 3 is a graphical illustration of an exemplary recommendation made by the processing unit of FIG. 2 via a graphical user interface (GUI) in accordance with embodiments;

FIG. 4 is a graphical illustration of an exemplary recommendation made by the processing unit of FIG. 2 via a graphical user interface (GUI) in accordance with embodiments;

FIG. 5 is a schematic illustration of an exemplary air pollution/allergen exposure measurement system in accordance with embodiments; and

FIG. 6 is a flow diagram illustrating an exemplary air pollution/allergen exposure measurement method in accordance with embodiments.

### DETAILED DESCRIPTION

Air pollution/allergen exposure tracking is a direct way to determine the health impact of pollution levels. However, indoor and outdoor air monitors have, to date, been separate with indoor monitors being room based, not connected to the location of the individual.

Thus, as will be described below, data from indoor and outdoor air monitors is combined to determine a type of exposure a person experiences in a given day and/or a duration of that exposure and quantities of pollution the person is exposed to during the day. This will then enable personalized air quality exposure monitoring and tailored recommendations. Additionally, individuals can select to monitor and receive notifications of exposure levels to specific pollutants or allergens, such as tree pollen, and pet dander.

An algorithm is provided that calculates the person's exposure to airborne pollutants over the course of a day. Personal air quality monitors are embedded on the person, a wearable device, and/or and in his/her home (and/or place of work). Using combinations of data sets from the personal air monitors, the person can track his/her daily exposure to air pollution/allergen or specific allergens. This tracking allows for recommendations to be made regarding avoiding pollutants or allergens and for establishing thresholds that issue alarms after being met. The algorithm makes use of wifi positioning and air monitors to determine exposure for the person.

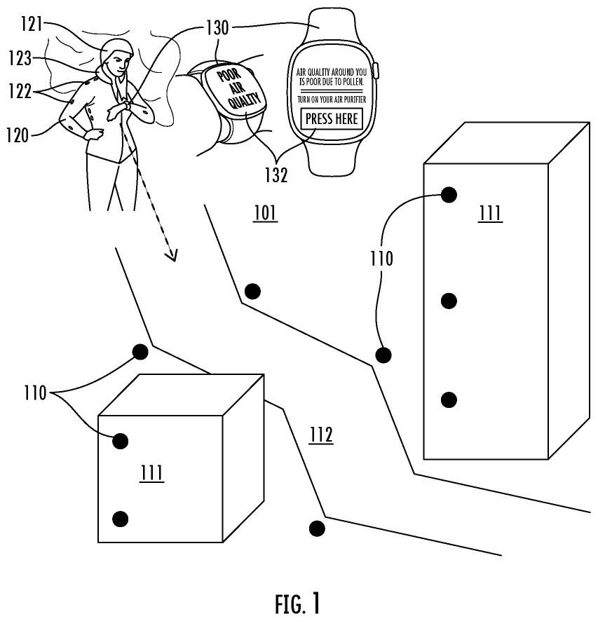

With reference to FIGS. 1 and 2, an air pollution/allergen exposure measurement system 101 is provided. The air pollution/allergen exposure measurement system 101 includes fixed air pollution/allergen measurement devices 110, a wearable article 120 to be worn by a user 121 and including a wearable air pollution/allergen measurement device 122, a mobile device 130 carried by the user 121 for providing location monitoring of the user 121 and a processing unit 201 (see FIG. 2). The processing unit 201 is coupled to or otherwise disposed in signal communication with the fixed air pollution/allergen measurement devices 110, the wearable air pollution/allergen measurement device 122 and the mobile device 130.

The fixed air pollution/allergen measurement devices 110 can be deployed in a defined indoor and/or a defined outdoor space through which the user 121 is movable. As examples, the fixed air pollution/allergen measurement devices 110 can be deployed in at least one of a building 111 through which the user 121 is movable and an outdoor space 112 through which the user 121 is movable. In any case, each of the fixed air pollution/allergen measurement devices 110 can be disposed and configured to measure or sense air a quality of air and/or a concentration of air pollutants within a range thereof. The wearable air pollution/allergen measurement device 122 can be disposed and configured similarly, though it is important to note that each of the fixed air pollution/allergen measurement devices 110 and the wearable air pollution/allergen measurement device 122 need not be disposed and configured to sense the quality of the air in a same manner and/or to sense same types of air pollutants. For example, some of the fixed air pollution/allergen measurement devices 110 can be disposed and configured as smoke detectors, others of the fixed air pollution/allergen measurement devices 110 can be disposed and configured as carbon monoxide (CO) detectors and the wearable air pollution/allergen measurement device 122 can be disposed and configured as a CO detector.

The wearable article 120 can be provided as a type of clothing to be worn by the user 121, such as, but not limited to, a jacket or a vest, and can include the wearable air pollution/allergen measurement device 122, one or more additional air pollution/allergen measurement devices and a selectively activatable air quality improvement element 123. The selectively activatable air quality improvement element 123 can be provided as a fan, a blower, a suction device or another similar device that can clear air from around the user 121. In any case, the selectively activatable air quality improvement element 123 is activatable by the user 121 or the processing unit 201.

The mobile device 130 includes a global positioning system (GPS) unit 131 (see FIG. 2), which is usable for location monitoring and tracking of the user 121 during a given time period, such as a day in the user's life. The mobile device 130 can include or be provided as at least one of a mobile computing device, a smartphone and a smartwatch with the processing unit 201 being embodied in the mobile device 130. In any case, the mobile device 130 can include a display by which a graphical user interface (GUI) 132 is displayed to the user. The processing unit 201 can be wirelessly coupled to the fixed air pollution/allergen measurement devices 110 and the wearable air pollution/allergen measurement device 122 and the mobile device 130 (where applicable) via any type of cell network or the internet.

With reference to FIG. 2, the processing unit 201 can be embodied in the mobile device 130 or can be remote from the mobile device 130 (in which case, the processing unit 201 can be wirelessly coupled to the mobile device 130). In either case, the processing unit 201 incudes a processor 210, such as a central processor or CPU, a memory unit 220 and a networking unit 230 by which the processor 210 is communicative with the fixed air pollution/allergen measurement devices 110, the wearable air pollution/allergen measurement device 122 and the mobile device 130 (where applicable). The memory unit 220 has executable instructions stored thereon that are readable and executable by the processor 210. When the executable instructions are read and executed by the processor 210, the executable instructions cause the processor 210 to operate as described herein.

With the processor 210 operating according to the executable instructions, the processing unit 201 is configured to determine the fixed air pollution/allergen measurement devices 110 that the user 121 has been within range of from the location monitoring by the GPS unit 131 of the mobile device 130. In addition, with the processor 210 operating according to the executable instructions, the processing unit 201 is configured to determine a quality of air surrounding the user 121 from readings of the wearable air pollution/allergen measurement device 122 and the fixed air pollution/allergen measurement devices 110 the user 121 has been within range of and to make recommendations via the GUI 132 to the user 121 for improving the quality of air surrounding the user 121 during the given time period.

With reference to FIG. 3 and in accordance with embodiments, the recommendations made by the processing unit 201 can include displaying a recommended route 301 via the GUI 132 for informing the user 121 as to how to move though the defined indoor and/or the defined outdoor space to experience improved air quality (i.e., to avoid pockets of unclean air that the processing unit 201 is aware of from readings of the fixed air pollution/allergen measurement devices 110).

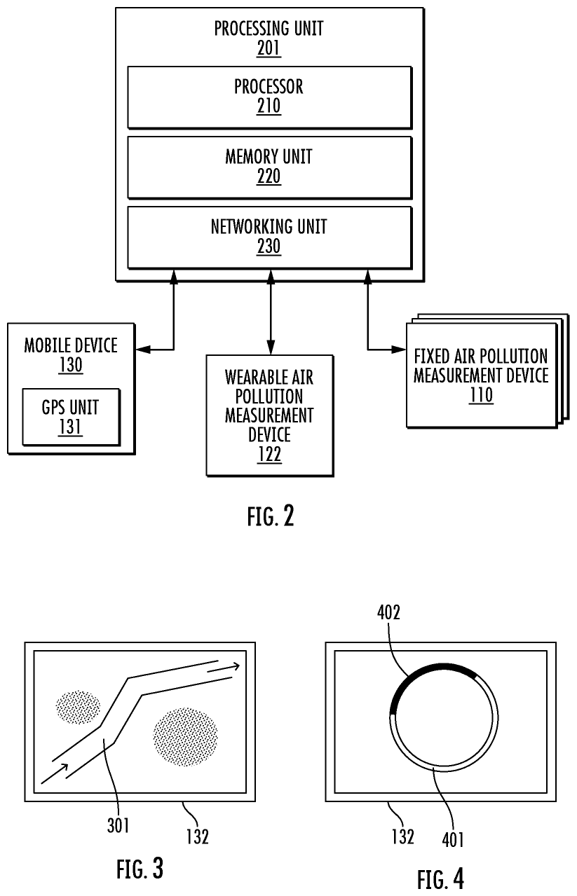

With reference to FIG. 4 and in accordance with embodiments, the recommendations made by the processing unit 201 can include displaying on the GUI 132 an air quality goal 401 for the user 121 along with an experienced air quality reading 402 for the user 121. As shown in FIG. 4, the air quality goal 401 and the experienced air quality reading 402 can be displayed as rings to be completed (or not completed) for the user 121 during the given time period.

Thus, it can be seen that the processing unit 201 is generally configured to help the user 121 to experience improved air quality during the given time period.

With reference to FIG. 5, an air pollution/allergen exposure measurement system 501 is provided and includes features which are similar to the described above and which need not be described in detail again. In particular, the air pollution/allergen exposure measurement system 501 includes a wearable article 510 and a processing unit 520. The wearable article 510 is to be worn by a user 511 and includes a wearable air pollution/allergen measurement device 512 and a selectively activatable air quality improvement element 513. The processing unit 520 can be embodied in at least one of a mobile computing device, a smartphone and a smartwatch carried by the user 511 and is (wired or wirelessly) coupled to the wearable air pollution/allergen measurement device 512 and the selectively activatable air quality improvement element 513. The processing unit 520 is configured to determine a quality of air surrounding the user 511 from readings of the wearable air pollution/allergen measurement device 512 and to selectively activate the selectively activatable air quality improvement element 513, which is also activatable by the user 511, accordingly for improving the quality of air surrounding the user 511. The processing unit 520 can be interacted with by the user 511 by way of a GUI 521.

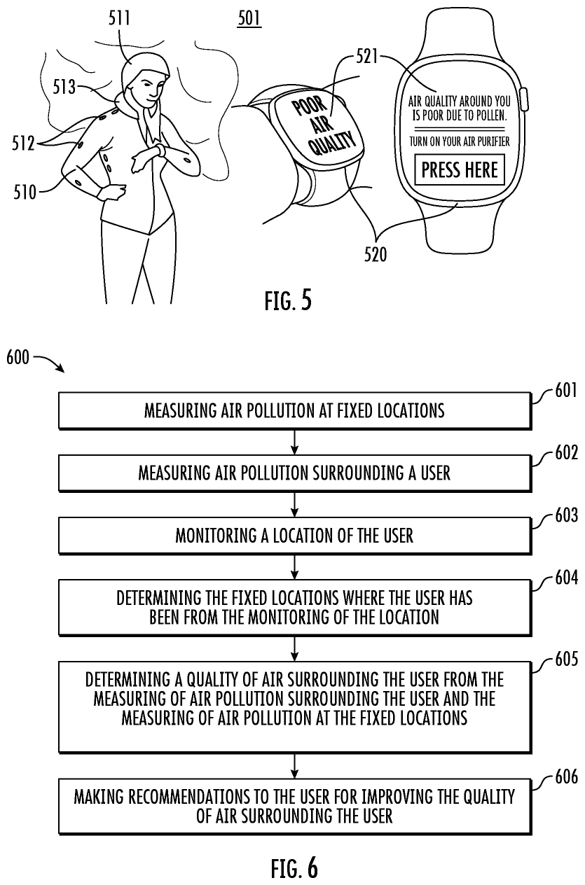

With reference to FIG. 6, an air pollution/allergen exposure measurement method 600 is provided. The method 600 can be executed by the features described above and includes measuring air pollution/allergen at fixed locations (601), measuring air pollution/allergen surrounding a user (602), monitoring a location of the user (603), determining the fixed locations where the user has been from the monitoring of the location (604), determining a quality of air surrounding the user from the measuring of air pollution/allergen surrounding the user and the measuring of air pollution/allergen at the fixed locations (605) and making recommendations to the user for improving the quality of air surrounding the user (606).

In accordance with embodiments, the determining of the quality of the air of operation 605 can be achieved by monitoring time and concentration of pollutants being monitored by one or more sensors. Other methods to determine the quality of the air of operation 605 could include a historical tracking of sensor data in the area, such as statistical analysis or linear regression of sensor data based on a geo-spatial reference point. The accuracy and localization can be improved by using digital twin for pollution models to provide more granular exposure assessment.

In accordance with embodiments, the making of recommendations to the user of operation 605 can include providing information to the user such as a pollutant type, an exposure time and a concentration monitoring time, all of which can also be used to determine the recommendation. An expert system algorithm can be constructed in two or more dimensions and can have at least two layers of decision logic to parse recommendations for single pollutant actions and multipollutant actions.

Technical effects and benefits of the present disclosure are the provision of methods and systems whereby an active and healthy user wears clothing and other items that provide personal air quality measurements within a close personal space. This active and healthy user would feel reassured with a watch application, a wearable device or a mobile application which will help monitor the air quality of their immediate environment.

The corresponding structures, materials, acts, and equivalents of all means or step plus function elements in the claims below are intended to include any structure, material, or act for performing the function in combination with other claimed elements as specifically claimed. The description of the present disclosure has been presented for purposes of illustration and description, but is not intended to be exhaustive or limited to the technical concepts in the form disclosed. Many modifications and variations will be apparent to those of ordinary skill in the art without departing from the scope and spirit of the disclosure. The embodiments were chosen and described in order to best explain the principles of the disclosure and the practical application, and to enable others of ordinary skill in the art to understand the disclosure for various embodiments with various modifications as are suited to the particular use contemplated.

While the preferred embodiments to the disclosure have been described, it will be understood that those skilled in the art, both now and in the future, may make various improvements and enhancements which fall within the scope of the claims which follow. These claims should be construed to maintain the proper protection for the disclosure first described.

What is claimed is:

1. An air pollution/allergen exposure measurement system, comprising:
at least one fixed air pollution/allergen measurement device;
a wearable article to be worn by a user and comprising a wearable air pollution/allergen measurement device;
a mobile device carried by the user for location monitoring of the user; and
a processing unit coupled to the fixed air pollution/allergen measurement device(s), the wearable air pollution/allergen measurement device and the mobile device,
the processing unit configured to determine the fixed air pollution/allergen measurement device(s) the user has been within range of from the location monitoring, to determine a quality of air surrounding the user from readings of the wearable air pollution/allergen measurement device and the fixed air pollution/allergen measurement device(s) the user has been within range of and to make recommendations to the user for improving the quality of air surrounding the user.

2. The air pollution/allergen exposure measurement system according to claim 1, wherein the fixed air pollution/allergen measurement device(s) is/are deployed in a defined space through which the user is movable.

3. The air pollution/allergen exposure measurement system according to claim 1, wherein the fixed air pollution/allergen measurement device(s) are deployed in at least one of a building through which the user is movable and an outdoor space through which the user is movable.

4. The air pollution/allergen exposure measurement system according to claim 1, wherein:
the wearable article comprises a selectively activatable air quality improvement element, and
the selectively activatable air quality improvement element is activatable by the user or the processing unit.

5. The air pollution/allergen exposure measurement system according to claim 1, wherein the mobile device comprises a global positioning system (GPS) unit for user location monitoring and tracking.

6. The air pollution/allergen exposure measurement system according to claim 1, wherein the mobile device comprises at least one of a mobile computing device, a smartphone and a smartwatch and the processing unit is embodied in the mobile device.

7. The air pollution/allergen exposure measurement system according to claim 1, wherein the processing unit is wirelessly coupled to the fixed air pollution/allergen measurement device(s) and the wearable air pollution/allergen measurement device.

8. The air pollution/allergen exposure measurement system according to claim 1, wherein the mobile device comprises a graphical user interface (GUI) by which the processing unit makes the recommendations to the user.

9. The air pollution/allergen exposure measurement system according to claim 8, wherein the processing unit displays, by way of the GUI, at least one of:
a recommended route for improved air quality by way of the GUI, and
an air quality goal for the user along with an experienced air quality reading for the user.

10. An air pollution/allergen exposure measurement system, comprising:
a wearable article to be worn by a user and comprising a wearable air pollution/allergen measurement device and a selectively activatable air quality improvement element; and
a processing unit coupled to the wearable air pollution/allergen measurement device and the selectively activatable air quality improvement element,
the processing unit configured to determine a quality of air surrounding the user from readings of the wearable air pollution/allergen measurement device and to selectively activate the selectively activatable air quality improvement element accordingly for improving the quality of air surrounding the user.

11. The air pollution/allergen exposure measurement system according to claim 10, wherein the selectively activatable air quality improvement element is activatable by the user or the processing unit.

12. The air pollution/allergen exposure measurement system according to claim 10, wherein the processing unit is embodied in at least one of a mobile computing device, a smartphone and a smartwatch carried by the user.

13. The air pollution/allergen exposure measurement system according to claim 12, wherein the processing unit is interacted with by the user by way of a graphical user interface (GUI) of the at least one of the mobile computing device, the smartphone and the smartwatch.

14. The air pollution/allergen exposure measurement system according to claim 10, wherein the processing unit is wirelessly coupled to the wearable air pollution/allergen measurement device and the selectively activatable air quality improvement element.

15. An air pollution/allergen exposure measurement method, comprising:
measuring air pollution/allergen at fixed locations;
measuring air pollution/allergen surrounding a user;
monitoring a location of the user;
determining the fixed locations where the user has been from the monitoring of the location;
determining a quality of air surrounding the user from the measuring of air pollution/allergen surrounding the user and the measuring of air pollution/allergen at the fixed locations; and
making recommendations to the user for improving the quality of air surrounding the user.

16. The method according to claim 15, wherein the fixed locations are deployed in a defined indoor or outdoor space through which the user is movable.

17. The method according to claim 15, further comprising selectively activating an air quality improvement element for the user.

18. The method according to claim 15, wherein monitoring of the location of the user comprises global positioning of the user.

19. The method according to claim 15, wherein the making the recommendations comprises making the recommendations via a graphical user interface (GUI).

20. The method according to claim 19, wherein the making the recommendations via the GUI comprises at least one of:
recommending a route for improved air quality, and
displaying an air quality goal for the user along with an experienced air quality reading for the user.
